(19) United States
(12) Patent Application Publication
(10) Pub. No.: US 2022/0184607 A1
(43) Pub. Date: Jun. 16, 2022

(54) Method and Apparatus for Rapid Virus Screening and Detection using a Canine

(71) Applicant: Stanley G. Van Meter, II, Lake Mary, FL (US)

(72) Inventor: Stanley G. Van Meter, II, Lake Mary, FL (US)

(21) Appl. No.: 17/553,561

(22) Filed: Dec. 16, 2021

Related U.S. Application Data
(60) Provisional application No. 63/126,052, filed on Dec. 16, 2020.
(60) Provisional application No. 63/126,060, filed on Dec. 16, 2020.

(51) Int. Cl. B01L 3/00; G01N 33/00; G01N 33/487

(52) U.S. Cl. CPC B01L 3/5029; G01N 33/0036; G01N 33/48778; G01N 2800/26; B01L 2300/042; B01L 2300/048; B01L 2300/069

(57) ABSTRACT

An individual is given a specialized swab which will be inserted in the mouth for gathering saliva samples. After successfully obtaining a saliva sample, a cover is slide down the shaft of the cotton swap toward the head or sample end. When the cover reaches the head or sample end the cover over the head or sample end of the swab. The swab is placed into a holder for sniff detection by the canine. Canines specifically trained to detect a virus, such as SARS-CoV-2, also known as SARS-Covid19, COVID19 or the novel corona virus, using a sniff test are deployed. Then a canine identifies a COVID19 sample, they alert the handle. Finally, using the unique identifier on the swab, staff are able to identify who has been identified as having COVID19 for further action.

### FEDERALLY SPONSORED RESEARCH

Not Applicable

### SEQUENCE LISTING OR PROGRAM

Not Applicable

### CROSS REFERENCE TO RELATED APPLICATIONS

Not Applicable

### TECHNICAL FIELD OF THE INVENTION

The presence invention relates generally to virus screening and rapid detection methods and apparatus. More specifically, the presence invention relates to using a specifically designed collection apparatus to enable rapid canine virus detection based on pathogen-specific odor.

### BACKGROUND OF THE INVENTION

The ongoing COVID-19 pandemic highlights the importance of fast and reliable testing for accurate identification of symptomatic and asymptomatic carriers to reduce spread of infection effectively. Current testing regimens usually require nasopharyngeal swabs applied by a trained person and a reverse transcription polymerase chain reaction test (RT-PCR) for pathogen identification.

Obtaining RT-PCR results is time consuming and can be cost-prohibitive, especially for developing countries, and is therefore currently often used in a targeted fashion, testing predominantly patients with COVID-19 specific symptoms.

There is therefore a need for an additional faster, reliable, non-invasive, and versatile screening tool, especially to identify asymptomatic and pre-symptomatic individuals.

### SUMMARY OF THE INVENTION

A method or process for using canines to quickly and readily evaluate individuals for the presence of a virus. Now referring to FIG. 1, the process is illustrated. In a first step an individual is given a specialized swab which will be inserted in the mouth for gathering saliva samples.

Next, after successfully obtaining a saliva sample, the individual or a medical assistant slides a cover down the shaft of the cotton swap toward the head or sample end. When the cover reaches the head or sample end, the individual or medical assistant closes the cover over the head or sample end of the swab. The cover is designed to protect the saliva samples from coming in contact with individuals or canines.

The individual or medical assistant next places the swab into a holder for sniff detection by the canine. Because of the design of the collection device, individuals or medical assistants can hold the swaps at their sides and not risk spreading any potential virus by contact. Canines specifically trained to detect a virus, such as SARS-CoV-2, also known as SARS-Covid19, COVID19 or the novel corona virus, using a sniff test are deployed. Then a canine identifies a COVID19 sample, they alert the handle.

Finally, using the unique identifier on the swab, staff are able to identify who has been identified as having COVID19 for further action.

### BRIEF DESCRIPTION OF THE DRAWINGS

The accompanying drawings, which are incorporated herein form a part of the specification, illustrate the presence invention and, together with the description, further explain the principles of the invention and to enable a person skilled in the pertinent art to make and use the invention.

FIG. 1 is a flow chart illustrating the process and method taught by the presence invention for using a canine in the detection of a virus.

FIGS. 2-4 illustrate the collection and detection apparatus as taught by the presence invention.

### DETAILED DESCRIPTION OF THE INVENTION

Several studies have proven the canines' extraordinary olfactory acuity to detect persons with infectious and non-infectious diseases like different types of cancer, malaria, bacterial, and viral infections with usually high rates of sensitivity and specificity.

A pathogen-specific odor that can be detected by dogs may be composed of specific patterns of volatile organic compounds (VOCs). Compared to bacteria, viruses have no own metabolism, and therefore VOCs are released by infected body cells as a result of metabolic host processes. Different technical approaches have used the detection of VOCs to discriminate infectious diseases successfully, but none is being used routinely in clinical practice.

As dogs can be trained quickly, the aim of the presence study was to test the concept of using dogs reliably and in real-time to discriminate between samples of SARS-CoV-2 infected patients and non-infected controls. This method could be employed in public areas such as airports, sport events, borders, or other mass gatherings as an alternative or addition to laboratory testing, thus helping to prevent further spreading of the virus or further outbreaks.

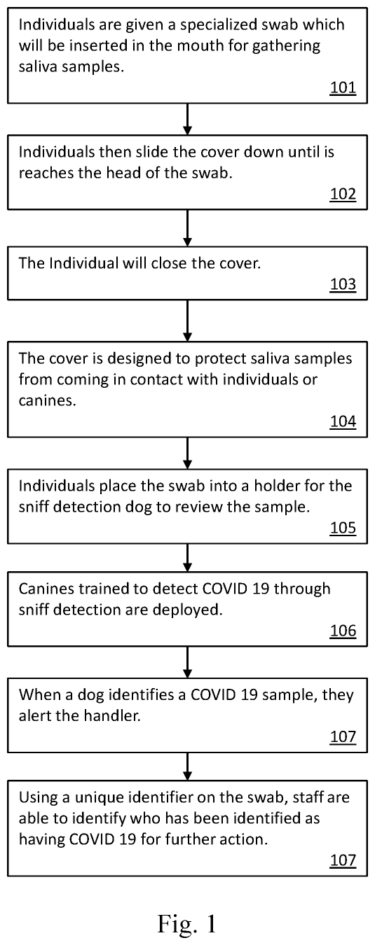

The presence invention is a method or process for using canines to quickly and readily evaluate individuals for the presence of a virus. Now referring to FIG. 1, the process is illustrated. In a first step an individual is given a specialized swab which will be inserted in the mouth for gathering saliva samples 101.

Next, after successfully obtaining a saliva sample, the individual or a medical assistant slides a cover down the shaft of the cotton swap toward the head or sample end 102. When the cover reaches the head or sample end, the individual or medical assistant closes the cover over the head or sample end of the swab 103. The cover is designed to protect the saliva samples from coming in contact with individuals or canines 104.

The individual or medical assistant next places the swab into a holder for sniff detection by the canine 105. Because of the design of the collection device, individuals or medical assistants can hold the swaps at their sides and not risk spreading any potential virus by contact.

Canines specifically trained to detect a virus, such as SARS-CoV-2, also known as SARS-Covid19, COVID19 or the novel corona virus, using a sniff test are deployed 106. Then a canine identifies a COVID19 sample, they alert the handler 107.

Finally, using the unique identifier on the swab, staff are able to identify who has been identified as having COVID19 for further action 107.

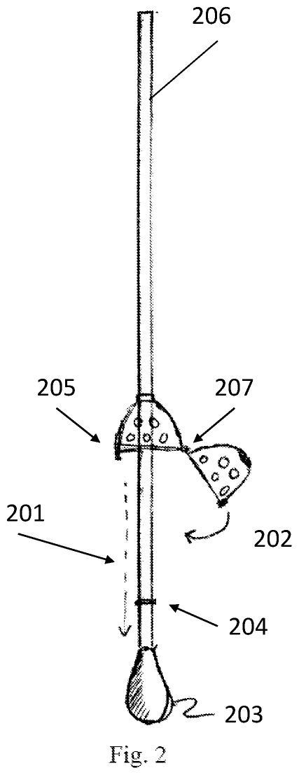

Now referring to FIGS. 2-4, the apparatus for enabling pathogen-specific odor detection by a canine is illustrated and taught. A medical swab comprising a thin stick or drawn plastic stick 206 and a sphere-like fiber lump 203 formed on at least one end of the stick; the fiber lump 203 has a fiber winding portion which is formed by winding a part of the fibers around the stick 206 and then is fixed to the stick 206 either by using an adhesive, or by tightening the fiber lump 203 containing a fibrillating cellulose without using an adhesive. The fiber lump 203 is herein referred to as the head or collection end of the swab.

Additionally, the fibers can also be chemically treated to enabled a color change when exposed to moisture. This is especially critical to ensure that when an individual or medical assistant takes a mouth swab that contact and transference of material has occurred between the moisture and cells located in an individual's mouth and the swab, helping to ensure testing validity and accuracy. The simple color change indicator allows an individual, who may be inexperienced with such testing procedures or a medical assistant to quickly and readily determine of the swabbing for cells and material in the mouth has been completed successfully.

On the stick 206 , a unique identifier sticker or tab is placed on the swab, the quick and easy unique identifier allows staff to identify who has been identified as having COVID19 or any virus for which detection is being sought for further action.

Also, on the thin stick 206, a cover 205 is slidably attached 201 and there is a slide stop located near the head or collection area 204. The cover 205 has an egg shape and split design where the cover is comprised of two equally sized pieces connected by a small hinge portion 207 allowing the cover 205 to be rotated between an open or closed position 202.

Opposing the hinge portion 207, clips 401 on the first and second cover portions can be engaged to secure the cover 205 in a closed position as shown in FIG. 4. In a closed positing the cover 205 creates a basket around the head or collection end 203.

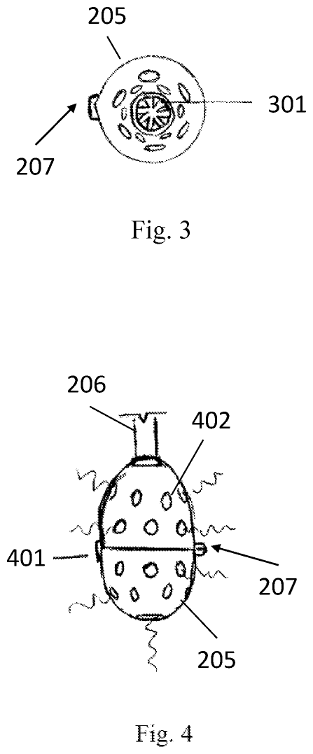

In an open position, a first, or bottom cover piece is further comprised of a hole of similar shape and diameter to that of the thin stick 206. The swab is connected to the cover 205 by placing the thin stick 206 portion down through the hole in the first/bottom cover piece. The hole of the first/bottom cover piece is comprised of one or more teeth 301 which provide gripe and securing means between the cover 205 and thin stick 206 but allow the cover 205 to slide along the stick 206 with minimal pressure as shown in FIG. 3.

As shown in FIG. 4, the cover 205 has a plurality of holes 402 which allow odor to escape from the cells or material collected on the swab. When in the closed position the clips 401 act as a clasp, which locks the cover 205 in place, creating a cage around the head or sample end 203. While the holes 402 allow odor to escape from the cells or material collected on the swab the cover or cage 205 prevents accidental contact by the individual, medical assistant, or canine once the sample has been collected the cover or cage securely closed.

Upon successfully completion of the collection step, an individual or medical assistant slides the cover toward the head or collection end until it engages or meets the slide stop. Once in this position the individual or medical assistant takes the sample for canine testing or in some instances a group of samples may be tested at one time or users may stand in a line with the sample to their side while one or more canines walks up and down the line testing each sample.

Although the presence invention has been described in considerable detail with reference to certain preferred versions thereof, other versions are possible. Therefore, the point and scope of the appended claims should not be limited to the description of the preferred versions contained herein.

As to a further discussion of the manner of usage and operation of the presence invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The embodiments of the invention in which an exclusive property or privilege is claimed are defined as follows:

1. A method for using canines to quickly and readily evaluate individuals for the presence of a virus:
an individual is given a specialized swab;
the swap is inserted in the mouth for gathering saliva samples;
after successfully obtaining a saliva sample, sliding a cover down the shaft of the cotton swap toward the head or sample end;
when the cover reaches the head or sample end, closing the cover over the head or sample end of the swab.

2. The method of claim 1, further comprising the step(s) of
placing the swab into a holder for sniff detection by the canine.

3. The method of claim 2, further comprising the step(s) of
providing canines specifically trained to detect a virus, such as SARS-CoV-2, also known as SARS-Covid19, COVID19 or the novel corona virus, using a sniff test are deployed;
exposing the canine(s) to one or more samples; and
identifying a positive sample; and
providing an alert.

4. The method of claim 3, wherein
the specialized swab is further comprised of a color changing chemical compound; and
the color changing compound changes to a color in the presence of moisture.

5. The method of claim 4, further comprising the step(s) of
the color changing compound changes to a color in the presence of moisture as a result of a correct saliva sample being taken.

6. The method of claim 4, further comprising the step(s) of
detecting a correct saliva sample being taken as a result of the color changing compound changing to a color.

7. The method of claim 4, wherein
the specialized swab is further comprised of a unique identifier.

8. The method of claim 4, further comprising the step(s) of
Assigning the unique identifier of the swap to an individual; and
identifying an individual using the unique identifier of the swap after a positive alter.
